(19) United States
(12) Patent Application Publication
(10) Pub. No.: US 2022/0236131 A1
(43) Pub. Date: Jul. 28, 2022

(54) SEATING SYSTEM WITH USER MONITORING CAPABILITIES AND METHODS FOR MONITORING USER

(71) Applicants: NATHAN P. CLEMENTE, Chicago, IL (US); THOMAS P. JENSEN, Eagle, ID (US); STEVEN D. MUSCHLER, Chicago, IL (US); RYAN RAMOS, Milwaukee, WI (US)

(72) Inventors: NATHAN P. CLEMENTE, Chicago, IL (US); THOMAS P. JENSEN, Eagle, ID (US); STEVEN D. MUSCHLER, Chicago, IL (US); RYAN RAMOS, Milwaukee, WI (US)

(21) Appl. No.: 17/588,064

(22) Filed: Jan. 28, 2022

Related U.S. Application Data
(60) Provisional application No. 63/142,753, filed on Jan. 28, 2021.

(51) Int. Cl. G01L 19/00; G16H 10/60; G05B 15/02; G05D 7/06

(52) U.S. Cl. CPC G01L 19/0092; G05D 7/0623; G05B 15/02; G16H 10/60

(57) ABSTRACT

A method for adjusting the pressure of one or more cushions in a human transport system is disclosed. The one or more cushions include a plurality of inflatable chambers and a plurality of pressure sensors associated with the cushions. The pressure sensors may be surface pressure sensors or chamber pressure sensors. A processor associated with the human transport system obtains pressure measurements from the plurality of pressure sensors. Based on the pressure measurements, the processor identifies one or more of the plurality of inflatable chambers whose pressure requirements adjustment. The processor then sends commands that cause the human transport system to adjust the pressure in the one or more inflatable chambers. Systems including cushions and processors are also disclosed.

### CROSS REFERENCE TO RELATED APPLICATIONS

This application is based on and claims priority to U.S. Provisional Application Ser. No. 63/142,753 filed Jan. 28, 2021, which is incorporated by reference herein in its entirety for all purposes.

### BACKGROUND OF THE INVENTION

Mobile seating systems such as wheelchairs are widely used to provide users with medical conditions the ability to move about. But wheelchairs have not typically been designed with the user in mind. Instead, traditional wheelchairs may be unintelligent, uncomfortable, and unhelpful to a user, a caregiver, a medical provider, and others. For example, some wheelchairs provide little more than a piece of fabric for a seat and back rest, providing little support to the user. In contrast, some wheelchairs provide rigid materials for a seat and back rest, causing discomfort to the user. And wheelchairs typically provide little functional utility other than mobility. For example, when a medical provider wants to run medical tests on the user, the user must be removed from the wheelchair and placed on a separate surface (such as a gurney or exam table). Wheelchairs with additional functionality are heavy and cumbersome to operate.

In addition, wheelchairs can cause more medical problems for a user. For example, sitting in a wheelchair can increase the risk of developing bed sores, skin tears, and infections. As another example, sitting in a wheelchair can exacerbate pain for users with hip, back, and leg problems. Existing wheelchairs do not adequately address user needs.

### SUMMARY OF THE INVENTION

The disclosed subject matter includes a system such as a mobility system, a mobile seating device, or a human transport device. The mobility system may be a system that assists the user in moving by themselves (i.e., without the assistance of another person), such as a wheelchair, an electric scooter, a rollator, or a walker. A mobile seating device may be any device that provides mobility while a user is seated such as a wheelchair or an electric scooter. A human transport device (e.g., a patient transport device) may be any device that assists with transporting a human. For example, the human transport device may be a wheelchair or a gurney designed to transport patients with medical conditions. Although the embodiments described herein will use the example of a wheelchair, the disclosed subject matter may be used with other mobility systems, mobile seating devices, and/or human transport devices. In some examples, the disclosed subject matter may also include stationary seating systems. For purposes of explanation, the phrase “seating system” will be used to collectively refer to mobility systems, mobile seating devices, human transport devices, and stationary seating systems where a user is oriented in any position including seated, prone or any position in between.

Seating systems in accordance with the disclosed subject matter may include one or more integrated sensors, including without limitation one or more pressure sensors, one or more temperature sensors, one or more heart rate sensors, and one or more moisture sensors. By integrating intelligent sensors that can read data within a modular ecosystem clinicians, caregivers, and family members can not only monitor their activities but can prescribe treatments that may lead to better health outcomes. This ecosystem of synthesized data may be sent to a centralized or accessible location, such as an electronic medical record system, to assist clinicians in their treatment plans.

The disclosed subject matter also includes systems including the disclosed mobility systems, mobile seating devices, human transport devices, and stationary seating systems and a backend system. The backend system may be configured to store information received from the disclosed seating systems, such as biometric sensor data. The backend system may also interface with other systems such as an electronic medical records system, a web portal, or a software application on a medical device to provide biometric sensor data to a user's medical provider, caregiver, or friends and family.

The disclosed subject matter also includes methods performed by, using, or related to the disclosed mobility systems, mobile seating devices, human transport devices, and/or stationary seating systems. Such methods may include methods for adjusting the pressure of one or more fluid-filled cushions (for example, gas-filled cushions) automatically or based on user feedback, methods for detecting an alert condition, methods for activating an alert based on detecting an alert condition, methods for providing non-biometric sensor data to a backend system and/or an electronic medical records system, methods for developing a treatment plan for a user based on sensor data received by an electronic medical records system, and the like. The backend system may be configured to store information received from the disclosed seating systems, such as pressure sensor data and seating system orientation. The backend system may also interface with other systems such as an electronic medical records system, a web portal, or a software application on a medical device to provide such information to a user's medical provider, caregiver, or friends and family.

The disclosed mobility systems, mobile seating devices, human transport devices, and/or stationary seating systems may be used by a user who may have health issues. The user may have a caregiver that provides assistance to the user. In some examples, the caregiver may be a trained medical professional such as a hired nurse, clinician, or therapist. In some other examples, the caregiver may be a family member or other person that does not have medical training. The user may also have a medical or other care provider (e.g., a primary care physician or another doctor or nurse, or a physical or occupational therapist). The medical provider may be different than the caregiver. In some embodiments, the disclosed system may provide medical providers and/or caregivers the ability to monitor the user and his or her health.

In some embodiments, the disclosed subject matter includes a method of controlling the pressure in a cushion. The method may include providing at least one cushion. The at least one cushion may have a plurality of inflatable chambers, which may be fluid-filled (e.g., gas-filled) chambers. The method may also include obtaining pressure measurements from a plurality of pressure sensors associated with the at least one cushion. The pressure sensors may include surface pressure sensors located on a surface of the cushion or chamber pressure sensors associated with individual inflatable chambers.

The method may further include identifying at least one inflatable chamber of the at least one cushion based at least in part on the pressure measurements. In some examples, the at least one inflatable chamber may be identified by determining that a pressure measurement associated with the at least one inflatable chamber meets or exceeds a threshold pressure value. In some examples, the at least one inflatable chamber may be identified by determining that a pressure measurement associated with the at least one inflatable chamber is above or below a pressure value identified for the inflatable chamber is a pressure profile. In some examples, the pressure profile may be a profile saved in memory that the user has previously used. In some other examples, the pressure profile may be a recommended profile based on user information such as height, weight, body measurements, and/or medical condition. In some examples, the at least one inflatable chamber may be identified based on a pressure map and/or a seat map associated with a cushion.

The method may further include adjusting a pressure of the at least one inflatable chamber. For example, when a pressure associated with the at least one inflatable chamber exceeds a threshold pressure value, the pressure of the at least one inflatable chamber may be decreased, e.g., by releasing some of the fluid (e.g., gas such as air) in the inflatable chamber. In some other examples, the pressure of the at least one inflatable chamber may be increased, e.g., by adding fluid (e.g., has such as air) to the inflatable chamber if the pressure is below a threshold value.

In some examples, the method may include receiving input requesting an adjustment of the pressure within the at least one cushion. In some examples, the method may include obtaining second pressure measurements from the plurality of pressure sensors in response to the input. Input may be provided by the user or another person, including a care giver, family member or friend. In some examples, the method may include identifying a set of one or more inflatable chambers based at least in part on the second pressure measurements and adjusting the pressure of the set of one or more inflatable chambers.

In some examples, the disclosed subject matter may include a seating system such as a wheelchair. The seating system may include at least one cushion, a plurality of pressure sensors, one or more processors, and an actuator. The at least one cushion may include a plurality of inflatable chambers. The plurality of pressure sensors may be positioned on the at least one cushion, for example on the surface of the cushion, inside the cushion and associated with individual inflatable chambers in the cushion, or inside the inflatable cushions. The one or more processors may be configured to receive pressure measurements from the pressure sensors and identify one or more inflatable chambers based on the pressure measurements. The actuator may be coupled to the one or more processors and may be configured to adjust a pressure in the one or more inflatable chambers, e.g., in response to a signal or command provided by the one or more processors.

In some examples, the disclosed subject matter may include a system including a seating system, a backend system including memory, and an electronic medical records system. The seating system may include a cushion with a plurality of inflatable chambers, a plurality of pressure sensors associated with the cushion, and one or more biometric sensors configured to sense biometric data of a user of the biometric device. The biometric sensors may include, for example, a blood oxygen sensor, a blood glucose sensor, a heart rate sensor, or a blood pressure sensor. The seating system is configured to adjust a pressure of one or more of the inflatable chambers based on pressure readings from the plurality of pressure sensors and to transmit the pressure readings and biometric data to the backend system. The backend system is configured to store the pressure readings and biometric data or transmit the pressure readings and biometric data to centralized database, such as an electronic medical records system. The electronic medical records system is configured to receive the pressure readings and biometric data of the user, receive information related to a treatment plan for the user, and transmit the information related to the treatment plan to the seating system or the backend system.

### BRIEF DESCRIPTION OF THE DRAWINGS

FIG. 1 is a block diagram of a circuit board for a seating system in accordance with embodiments of the disclosed subject matter.

FIG. 2 is a flowchart describing a method for adjusting the pressure of a cushion on a seating system in accordance with embodiments of the disclosed subject matter.

FIG. 3 is a flowchart describing a method for adjusting the pressure of a cushion of a seating system in accordance with the disclosed subject matter.

FIG. 4 is a state diagram showing the states associated with adjusting the pressure of a cushion on a seating system in accordance with the disclosed subject matter.

FIG. 5 is a flowchart describing a method for activating an alert in accordance with embodiments of the disclosed subject matter.

FIG. 6 is a block diagram of a networked system in accordance with embodiments of the disclosed subject matter.

FIG. 7 is a flowchart describing a method for setting up a seating system in accordance with embodiments of the disclosed subject matter

FIG. 8 shows screenshots of an interface of a software application in accordance with embodiments of the disclosed subject matter.

FIG. 9 is a flowchart describing a method for communicating with a medical provider computer system in accordance with embodiments of the disclosed subject matter.

FIG. 10 is a block diagram of one embodiment of the surface of an armrest of a seating system in accordance with embodiments of the disclosed subject matter.

FIG. 11 is a diagram of one embodiment of a cushion that may be used with a seating system in accordance with embodiments of the disclosed subject matter.

### DETAILED DESCRIPTION

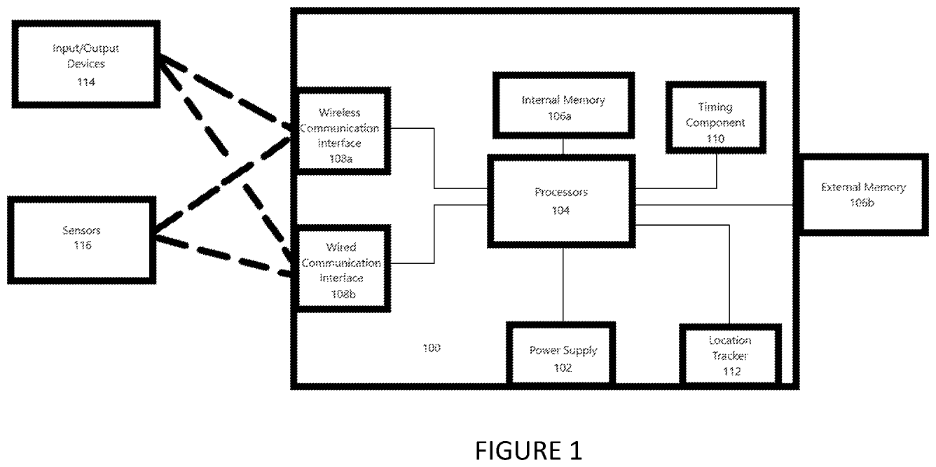

FIG. 1 is a block diagram of a circuit board 100 for a seating system in accordance with embodiments of the disclosed subject matter. The circuit board 100 may include a power supply 102, one or more processors 104, memory 106, communication interfaces 108, a timing component 110, and a location tracker 112. The circuit board may be, for example, a printed circuit board (PCB). Although FIG. 1 shows components located on the circuit board 100, one or more of these components may be moved off of the circuit board 100 without departing from the scope of the disclosed subject matter.

In some examples, the seating system may be a wheelchair. In some examples, wheelchair may be a manual wheelchair. In other examples, the wheelchair may be a power wheelchair that is propelled by electrical power. In some embodiments, the wheelchair may include a motor or other propulsion system, which may be stored underneath the seat portion of the wheelchair. In some other examples, the seating system may be a gurney.

The circuit board 100 may include a power supply 102 that provides power to seating system. The power supply 102 may include, for example, a battery such as a rechargeable battery. In some embodiments, the rechargeable battery could be connected to the circuit board 100 but be located in another location such as underneath the seat portion of a wheelchair. In some examples, the power supply 102 may include an interface where an external power supply may be connected to recharge the rechargeable battery. For example, the interface may provide a connection for an electrical cord that can be plugged into a wall outlet to allow the rechargeable battery to be recharged using the power grid. In some examples, the power supply 102 can include or be connected to a generator that can provide a power source for the seating system in an emergency. In some examples, the power supply 102 can include or be connected to photovoltaic cells that may be positioned on the seating system.

The circuit board 100 may also include one or more processors 104. The processors 104 may be configured to execute instructions stored in one or more memories 106 of the seating system. For example, the processors 104 may be configured to execute instructions to carry out any of the operations of the seating system as described herein, including but not limited to adjusting the pressure of a cushion based on pressure sensor measurements and/or user input, detecting an alert condition based on sensor measurements, communicating alert conditions to remote devices, and transmitting sensor data to a backend system for storage.

The circuit board 100 can include one or more memories 106. The one or more memories 106 can include volatile memory and/or non-volatile memory. In some examples, the one or more memories 106 may include internal memory 106a (which may be referred to as internal memory units or IMU) such as, for example, RAM, ROM, or flash memory. In some examples, the one or more memories 106 may also include external memory 106b, such as a data storage device (e.g., an SD card) that may be removably coupled to a memory interface of the seating system (e.g., an SD card slot). In some other examples, the external memory 106b may include embedded storage devices such as hard disks or solid-state drives. The one or more memories 106 may be communicably connected to the processors 104 such that the processors 104 can read from and write to the memories 106 as needed.

The circuit board 100 may further include one or more communication interfaces 108 that allow communication between the processors 104 and external systems.

In some embodiments, the circuit board 100 may include one or more wireless communication interfaces 108a that allow the seating system to communication with other components of the seating system and/or external systems over a wireless connection. The wireless communication interfaces 108a may include one or more of a Bluetooth communication unit including a Bluetooth antenna, a WiFi communication unit including a WiFi antenna, a cellular communication unit including a cellular antenna, an infrared communication unit, or any other type of wireless communication unit known in the art.

In some embodiments, the circuit board 100 may include one or more wired communication interfaces 108b that allow the processors 104 to communicate with other components of the seating system or to communicate with external systems including input devices. For example, the wired communication interfaces 108b may include one or more Universal Serial Bus (USB) ports. Other wired communication protocols may also be used, including for example Display Serial Interface (DSI), Inter-Integrated Circuit (I2C), or any other type of wired communication protocol as known in the art.

The circuit board 100 may include a timing component 110 such as a real time clock (RTC). The timing component may be used for various purposes, including for example to measure time for use in determining a user's heart rate in beats per minute, measuring the time duration a user is positioned in or on the seating system, and measuring the time duration the seating system is positioned in a specific orientation.

The circuit board 100 may include a location tracker 112. The location tracker 112 may be a global positioning system (GPS) unit. The GPS unit may include a GPS antenna. In some examples, the location tracker 112 may use another global navigation satellite system (GNSS) such as, for example, the Galileo system. The location tracker 112 may be used to locate the seating system if the seating system is lost, if the caregiver loses track of the user, or if the caregiver, medical provider, or a family member or friend of the user wants to check in on the location of the user. For example, the location tracker 112 may allow a caregiver to track the location of the seating system through a mobile application on the caregiver's device. The location tracker 112 may be used in combination with a weight sensor and/or pressure sensor to confirm that the user is still in the seating system. Alternatively, if the user is no longer in the seating system, historical data from the location tracker 112 may be combined with historical data from the weight sensor and/or pressure sensor to determine where the seating system was located when the user exited the seating system.

By using the communication interfaces 108, the processors 104 may be able to communicate with other components of the seating system and/or external systems. For example, the communication interfaces 108 may provide communications between the processors 104 and one or more input/output devices 114 and/or one or more sensors 116.

The input/output devices 114 may allow the user, caregiver, medical provider, or another interested party to provide information to or receive information from the seating system. For example, the input/output devices 114 may include one or more input devices such as a keyboard, a touchscreen, a joystick, microphone, or the like. For example, the user or caregiver may use a keyboard or a touchscreen to assist in the setup process, to control the operation of the seating system, to request information about the status of the user or the operation of the seating system, and/or to input information, including settings, into the seating system. In some examples, the seating system may include a joystick or other assistive technology device that allows the user to steer the seating system. In some examples, the seating system may include a microphone that allows for voice commands using voice recognition technology as known in the art, which may be beneficial if the user has a medical condition that prevents them from manually inputting information. In some examples, the seating system may include an input panel or touch screen, e.g., on one or both armrests of a wheelchair.

In some examples, the input/output devices 114 may include one or more output devices such as a display screen or a speaker. For example, the user and/or caregiver may use a display screen (e.g., an LCD display) to review requested information about the status of the user (e.g., to monitor the user's health) and/or the operation of the seating system. In some examples, the seating system may include one or more speakers to provide audio information to the user and/or caregiver, which may be beneficial if the user has a medical condition that prevents them from viewing visual (e.g., textual) information on a display screen. In some examples, the seating system may include one or more lights or other visual indicators that may provide information to the user and/or caregiver (e.g., lights that indicate the power status of the seating system and/or whether an alert condition exists).

In some examples, the input/output devices 114 may include one or more remote computing devices. For example, the input/output devices 114 may include a remote computing device controlled by a medical provider through which the medical provider can access medical records stored in the one or more memories 106 and/or through which the medical provider can store information to the one or more memories 106. For example, the medical provider may be able to set or update threshold values for emergency conditions or alerts based on the user's medical condition. In some examples, the input/output devices 114 may include a software application running on a mobile device that allows the caregiver, medical provider, or other interested party (e.g., family member or friend) to monitor the user's condition.

In some examples, the processors 104 may be configured to send alerts to one or more specified external systems when a predetermined condition occurs. The condition may be an emergency or non-emergency event. An emergency or non-emergency condition may occur based upon sensed biometric information or sensed seating system information. An example of an emergency condition includes but is not limited to a condition of the user (e.g., heart rate) or a condition of the seating system (e.g., an overturned seating system). An example of a non-emergency condition includes but is not limited to a condition of the seating system (e.g., low power storage). The processors 104 may determine that an emergency or non-emergency condition exists based on data received from sensors 116. For example, the processors 104 could be configured to send an alert to one or more of the following external systems when a predetermined condition occurs: one or more computing systems associated with the caregiver (e.g., a display screen connected to the seating system or a mobile communication device associated with the caregiver), one or more computing systems associated with the medical or other care provider (e.g., a computer at the medical provider's office or a mobile communication device associated with the medical provider), and/or one or more computing systems associated with family members or other individuals specified by the user (e.g., a mobile device associated with the user's child or spouse, or a close friend).

The sensors 116 may include one or more biometric sensors. The sensors 116 may be integrated with the seating, removably attached to the seating system (via a wired interface such as a USB port), or in wireless communication with the seating system.

For example, the sensors 116 may include a sensor that measures a user's blood oxygen level such as a pulse oximeter. In some examples, the pulse oximeter may be located on the seating system. For example, the pulse oximeter may be integrally formed on an armrest of the seating system. In other examples, the pulse oximeter may be removably connected to the seating system via a communication interface. In some other examples, the pulse oximeter may communicate with the seating system via wireless communications.

In some examples, the sensors 116 may include a heart rate sensor. The heart rate sensor may be, for example, an ECG or EKG sensor. In some other examples, heart rate sensor may provide raw information (e.g., voltage readings) and the heart rate may be determine based on the timing component 110. The heart rate sensor may include one or more electrical leads integrated into the seating system, e.g., in armrest, the seat portion, the back portion, and calf pads of the seating system.

In some examples, the sensors 116 may include a temperature sensor. The temperature sensor may include thin film sensors integrated into the seating system, e.g., in the seat portion, back portion, or armrest of the seating system. The temperate sensors may be configured to monitor body temperature averages over a period of time.

Other types of biometric sensors may also be used in accordance with the disclosed subject matter. For example, the sensors 116 may include a blood pressure sensor, a blood glucose sensor, or any other biometric sensor as known in the art. The biometric sensors may be integrated with the seating system, removably attached to the seating system, or wirelessly connected to the seating system.

The sensors 116 may also include a sensor for measuring the weight of the user. This sensor may be located on or under the seat of the seating system. The sensors 116 may also include one or more pressure sensors, which may be located on the seat portion of the seating system. The pressure sensors may be, for example, load cells or other transducers. The sensors 116 may also include sensors for measuring the speed and/or orientation of the seating system. For example, the sensors 116 may include a gyroscope for measuring the orientation of seating system. The sensors 116 may also include one or more motion sensors. The sensors 116 may include a sensor that measures the ambient temperature where the seating system is located.

In some examples, the sensors 116 may include a moisture sensor. In some examples, a cushion of the seating system may include thin film sensor elements to allow the seating system to detect moisture on the surface of the cushion, which may indicate a medical or comfort problem for the user. In some examples, the seating system may identify one or more areas of potential danger for skin breakdown, e.g., bed sores, skin tears, or infections based on the moisture sensor and/or pressure sensors. For example, the processors 104 may identify a location of potential danger when a moisture measurement at the location meets or exceeds a moisture threshold and a pressure measurement at the location meets or exceeds a pressure threshold. The seating system may communicate information identifying the location to the caregiver or medical or other care provider to assist them in caring for and providing medical treatment to the user.

One or more of the processors 104 may be configured to receive data from the one or more sensors 116 and control the operation of other electronic elements of the seating system based on the received data. For example, the processors 104 may display an alert on a display screen or provide output to other output devices based on the data received from the one or more sensors 116. In some other examples, the processors 104 may stop the motion of the wheelchair based on data received from a weight sensor indicating that the user is no longer seated in the seating system. The processors 104 can receive data gathered by the one or more sensors 116 and store the data in memory 106. The processors 104 also can retrieve data from one or more of the memory units 106. The one or more memory units 106 may be configured to store one or more default or configurable settings of the seating system such as the ranges associated with an alert condition for one or more sensors 116. The one or more memory units may also be configured to store historical sensor readings. In some examples, the one or more memory units may be configured to store historical sensor readings for a set period of time, e.g., two weeks. In some other examples, the one or more memory units may store historical sensor readings only until that information is transferred to a backend system. Such information may be transferred to the backend system for storage in real-time, at pre-determined intervals (e.g., every minute or every hour), or on-demand.

One or more features of the seating system may be controlled based at least in part on the sensor data. For example, the one or more memories 106 may include instructions that will cause the processor to perform certain actions if a condition occurs. For example, the seating system may automatically stop the movement of the seating system (e.g., by shutting down the motor or other propulsion system, or applying an electronic brake) if the detected weight suddenly drops to zero, indicating that the user is no longer in the seating system, or may be configured to sound an alarm or issue an alert to the caregiver, medical provider, or another person if a gyroscope indicates that the seating system has overturned.

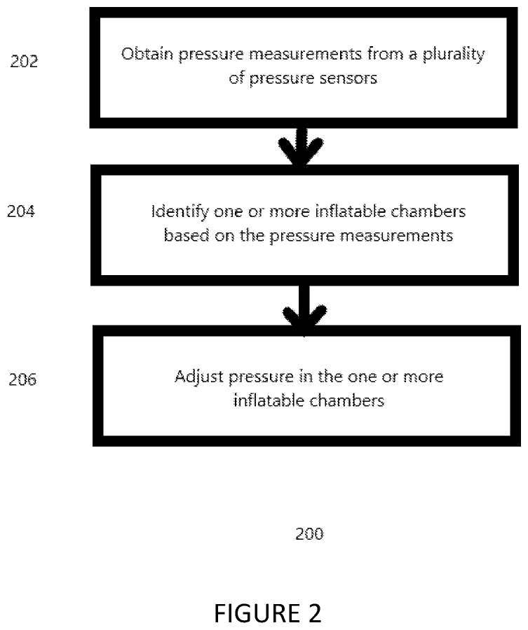

FIG. 2 is a flowchart 200 describing a method for adjusting the pressure of a cushion on a seating system in accordance with embodiments of the disclosed subject matter. The cushion may include one or more inflatable chambers. For example, the cushion may include multiple bladders. The bladders may be, for example, insert molded, co-molded, or otherwise formed from synthetic materials which may be sealed using solvents, heat, or other known sealing methods. In some examples, the cushion may include separate chambers (e.g., bladders) formed in different patterns. The patterns may vary based on the user's anatomy, the user's age, the user's gender, or the user's medical condition. In some examples, the bladder pattern may be subdivided into a lateral left portion and a lateral right portion, each with multiple bladders, or it may comprise a concentric circle pattern, wherein each circle or annular ring has one or more bladder sections. In some examples, each inflatable chambers may be approximately the same size and shape. In other examples, each inflatable chamber may have a different size or shape. However, the disclosed subject matter may be used with any configuration of inflatable chambers or bladders.

In embodiments of the disclosed subject matter, the seating system may include a fluid-filled (e.g., air-filled or liquid-filled) cushion. The cushion may be located in the seat area of the seating system and/or on the back of the mobile seating system (i.e., the area of the seating system against which the user rests his or her back during operation of the seating system). For example, the cushion may be located on seat portion and/or back portion of a seating system such as a wheelchair. Fluid-filled (e.g., gas-filled) cushions may alternatively or additionally be located on any area of the seating system against which a portion of the user's body may rest, either when the user is seated, lying prone, or otherwise using the seating system as described herein. In some examples, the seating system may be a gurney and a majority or even the entirety of the gurney may be covered by a fluid-filled cushion with one or more inflatable chambers as described herein. The cushion may be integrally formed with a surface. In other examples, the seating system may be configured to accept one or more cushions and each cushion may be provided with one or more interfaces for an electrical and/or data connection and an air or other gas connection. In some other examples, the cushion may be configured to operate independently (e.g., the cushion may have its own separate power source) but may be paired with the seating system via a wireless connection.

In some examples, the seating system may include a removable portion that may be transferred between different locations, e.g., between a first wheelchair and a second wheelchair or between a wheelchair and another seating apparatus (e.g., a seat of an airplane for use during travel). The removable portion of the seating system may include one or more cushions as described herein. In some examples, the removable portion may include a memory that stores user settings and/or software instructions for the removable portion (e.g., user pressure preferences for a fluid-filled cushion). For example, the memory may include instructions for adjusting the pressure of the cushion based on pressure readings in accordance with FIG. 2. In some examples, the removable portion may include a wireless communication module or unit (e.g., a Bluetooth unit including a Bluetooth antenna or a WiFi unit including a WiFi antenna) such that the user can control the removable portion remotely (e.g., to adjust the pressure of the cushion and/or request massage functionality as disclosed herein).

The method described in flowchart 200 begins with obtaining pressure measurements from a plurality of pressure sensors located on and/or integrated with the cushion at 202. In some examples, the pressure sensors may be surface pressure sensors that measure the pressure at a surface on the surface of the cushion. For example, the surface pressure sensor may be an electrical pressure sensor that covers the seating side of the cushion. In some other examples, the pressure sensors may be chamber pressure sensors that measure the pressure of the air or other gas in the inflatable chambers of the cushion. The chamber pressure sensors may be situated outside or inside each chamber and measure the pressure of the fluid located therein. In some other examples, the pressure sensors may be chamber pressure sensors that measure the pressure of the air or other fluid in the inflatable chambers. In some embodiments, the cushion may include both surface pressure sensors and chamber pressure sensors. Use of both types of sensors may provide additional data and may allow the seating system to better identify the user's comfort level and determine what adjustments may be required to reduce high pressure and ease discomfort. In some embodiments, the cushion may also include a water-resistant or moisture-resistant barrier to protect the pressure sensors.

In some examples, the seating system may constantly monitor the pressure measurements at the plurality of pressure sensors. In some other embodiments, the one or more pressure sensors may be coupled to a processor programmed to request pressure measurements from the pressure sensors on a periodic basis (e.g., every 30 seconds or every five minutes). In some other examples, the one or more pressure sensors may be coupled to a processor programmed to request pressure measurements in response to a request from the user, caregiver, medical provider, or other interested party.

The system may then identify one or more inflatable chambers based at least in part on the pressure measurements at 204. The inflatable chambers may be associated with one or more pressure measurements that meet or exceed a threshold value. For example, the data obtained from each pressure sensor may include a pressure measurement and a pressure sensor identifier. The system may check whether the pressure measurement meets or exceeds a threshold value. If so, the system may identify one or more inflatable chambers associated with pressure sensor indicated by the pressure sensor identifier. In some examples, surface pressure sensors may be associated with a first threshold value while chamber pressure sensors may be associated with a second threshold value. In some examples, the system may identify the one or more inflatable chambers based at least in part on a medical condition associated with the user. For example, if the user is a paraplegic, the system may identify the one or more inflatable chambers based on the surface pressure sensors and may not use the chamber pressure sensors to identify the one or more inflatable chambers.

In some examples, the threshold value may be a maximum pressure. In some examples, the maximum pressure may be a value selected by the user's medical provider based on the user's medical condition. For example, if the user has hip problems, the support provided to the hips (and the corresponding pressure for the inflatable chambers supporting the hips) may vary based on the nature of those problems.

In some examples, the seating system may store a data structure including a correlation between pressure sensors and inflatable cushions in memory. In some examples, the data structure may be a seat map that identifies the locations of the pressure sensors and the inflatable chambers. In some other examples, the data structure may be a table that identifies one or more inflatable cushions associated with each pressure sensor identifier. For chamber pressure sensors, the table may identify the inflatable chamber in which the pressure sensor is located. For surface pressure sensors, the table may identify one or more inflatable chambers that are located underneath the pressure sensor or otherwise associated with the pressure sensor. The system may identify the one or more inflatable chambers by looking up the inflatable chambers associated with a pressure sensor identifier.

In some other examples, the pressures sensors may be deployed in a configuration such that a pressure map can be generated from the readings from the pressure sensors. In such a configuration, high or low pressure readings may be identified (e.g., measured and/or calculated based on the measurements) not only at the location of the pressure sensors but also at locations between the pressure sensors. The system may identify the one or more inflatable chambers based at least in part on the pressure map and a seat map. For example, the system may identify one or more locations on the cushion associated with high pressure readings based on the pressure map, and then identify one or more inflatable chambers corresponding to the one or more locations based on the seat map.

The system may then adjust the pressure for each of the one or more inflatable chambers at 206. For example, the system may decrease the pressure in each of the one or more inflatable chambers to decrease the pressure on the user.

In some examples, the system may further store the pressure measurements in memory. In some examples, the system may compare the pressure measurements from chamber pressure sensors to expected chamber pressures for leak detection. If a leak is detected, the seating system may provide an alert to the user, the caregiver, or another person.

In some examples, the pressure in the inflatable chambers of the cushion may be controlled according to instructions stored in a memory of the seating system or remotely. In some examples, the pressure in the inflatable chambers may additionally or alternatively be controlled by instructions provided by a user or other person. For example, the seating system may include an interface device or a control panel that allows the user or caregiver to increase or decrease the pressure in the individual inflatable portions. In other examples, the seating system may be controllable through communications interfaces (e.g., a Bluetooth or WiFi receiver) by a remote person such as a medical provider using, e.g., an application on a mobile phone or a computer.

In some examples, the processor may detect that the user has shifted based on changes in pressure across all sensors. The processor may adjust the pressures in the inflatable chambers in response to this change of position. The processor may adjust the pressures in the inflatable chambers based at least in part on historical data.

In some other examples, the pressure of each inflatable system may be adjusted based on feedback from the user. For example, the user or caregiver may be able to indicate that the user is feeling pain and the processor may be configured to adjust the pressure in one or more inflatable portions based on that feedback.

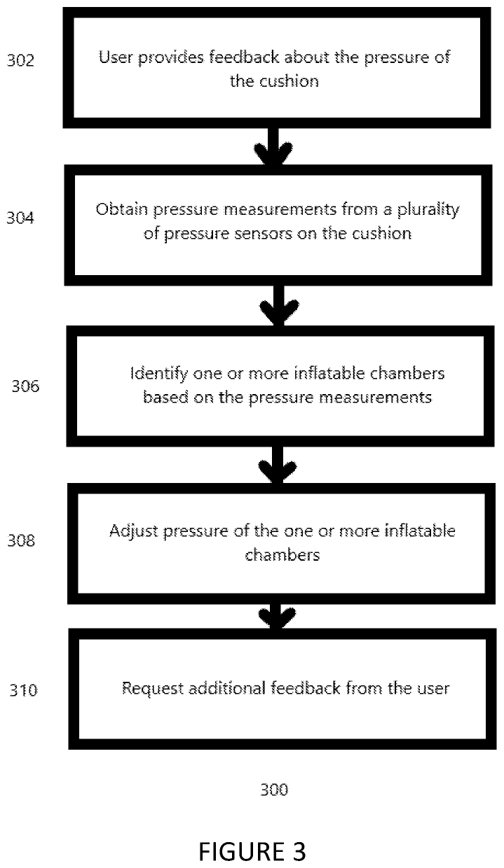

FIG. 3 is a flowchart 300 describing a method for adjusting the pressure of a cushion of a seating system in accordance with the disclosed subject matter.

The user may provide feedback about the pressure of the cushion at 302. In some examples, the user may provide the feedback through an input device on the seating system. For example, the input device may be a button or touch screen provided to allow the user to express discomfort. In some other examples, the user may provide the feedback through a software application such as a smartphone app. The software application may transmit the feedback to the seating system directly (e.g., via wireless communication) or indirectly (e.g., through a backend system). In some examples, the feedback may include an indication that the user is uncomfortable. In some other examples, the feedback may include additional information about the discomfort, such as an indication of a body part where the user is feeling discomfort or an area of the cushion where the user is feeling discomfort. For example, the user may indicate an area on a seat map corresponding to the location where the user is feeling discomfort. The seating system may receive the feedback provided by the user.

The seating system may obtain pressure measurements from a plurality of pressure sensors on and/or integrated with the cushion at 304. In some examples, the seating system may obtain pressure measurements from all of the pressure sensors on the cushion. In some other examples, the seating system may obtain pressure measurements from a subset of the pressure sensors on the cushion. For example, if the user has identified an area of the seat where they are feeling discomfort, the seating system may obtain pressure measurements only from the pressure sensors in that area of the cushion.

The seating system may identify one or more inflatable chambers at 306. The one or more inflatable chambers may be identified based on the obtained pressure measurements. For example, the seating system may identify one or more inflatable chambers associated with the highest pressure measurements of the obtained pressure measurements. In some other examples, the seating system may identify one or more inflatable chambers associated with the lowest pressure measurements of the obtained pressure measurements. In some other embodiments, if the user has identified a location associated with the discomfort, the seating system may identify an inflatable chamber associated with a highest or lowest pressure measurement in proximity to the identified location (e.g., within about one inch, within about two inches, or within about three inches of the identified location). In some embodiments, if the user has identified a body part associated with the discomfort, the seating system may identify an inflatable chamber based in part on the body part. For example, if the user identifies hip pain or numbness, the seating system may store information about the locations on a pressure map that are most likely to cause hip pain or numbness and may identify one or more inflatable chambers near those locations.

In some examples, the seating system may identify the one or more inflatable sensors based on stored pressure settings. For example, the seating system may store one or more profiles that specify pressure values for each of the inflatable chambers. In some examples, the one or more profiles may include a first profile (e.g., associated with a firm cushion) and a second profile (e.g., associated with a soft cushion). The user may identify one of the known settings in providing feedback at 302. The seating system may compare the pressure values associated with the selected profile with the pressure values measured by the chamber pressure sensors to identify one or more inflatable chambers that require an increase in pressure and/or one or more inflatable chambers that require a decrease in pressure.

In some examples, the seating system may determine a recommended profile based at least in part on user information. The user information may be information entered by a user such as height and weight. In some embodiments, the user information may additionally or alternatively include body measurements captured by a camera or video camera on the seating system. The seating system may determine recommended pressure values for each inflatable chamber based at least in part on the user information. The seating system may then compare the recommended pressure values with the pressure values measured by the chamber pressure sensors to identify one or more inflatable chambers that require an increase in pressure and/or one or more inflatable chambers that require a decrease in pressure.

The seating system may then adjust the pressure of the one or more inflatable chambers at 308. In some examples, the seating system may increase the pressure in the one or more inflatable chambers by adding more fluid to the inflatable chambers. In some other examples, the seating system may decrease the pressure in the one or more inflatable chambers by allowing fluid to escape from the one or more inflatable chambers.

The seating system may then request additional feedback from the user at 310. For example, the seating system may request user feedback on whether the user is still uncomfortable. The user may indicate that they are no longer in discomfort, in which case the process ends. Alternatively, the user may indicate that they are still in discomfort, in which case the process may return to step 304 and further adjustments to the pressure of the cushion may be made. The seating system may also request user feedback on whether the user is more comfortable or less comfortable than before the adjustments were made. If the user indicates they are less comfortable, the seating system may reverse the pressure adjustments before returning to step 304. If the user indicates they are more comfortable, the seating system may continue to make further pressure adjustments on top of the initial pressure adjustments. The process may continue until the user indicates they are comfortable.

In some embodiments, the pressure of each inflatable portion may be varied in time to provide a massage functionality. By controlling the variance of pressure in each inflatable portion, the seating system may provide an undulation. The frequency of the undulation may be programmed to a certain rate or may be selected randomly or semi-randomly, and in some cases may be varied by the user, caregiver, or medical provider. This functionality may also be activated and deactivated at programmed, random, or semi-random intervals, or activated on demand by the user, caregiver, or medical provider. This feature may be provided and/or implemented for comfort and/or medical reasons, such as providing pain relief.

In some examples, the seating system may also adjust the pressure in the inflatable chambers in accordance with an operating mode. For example, when the user is attempting to get out of the seating system, either on their own or with the assistance of another such as a caregiver, the seating system may provide a higher pressure on one side of the cushion (e.g., the back) and a lower pressure at the other side of the cushion (e.g., the back) to make it easier for the user to slide out of the chair by providing a downward slope or make it easier for the caregiver to grasp the user and assist them out of the chair. In other examples, such as where the inflatable chambers are formed in a concentric circle pattern, the seating system may provide a higher pressure in the center of the cushion and a lower pressure on the outer portions of the cushion. When the user is attempting to get back into the seating system, the pressure gradient may be provided in the opposite direction. In still other examples, the pressures may be varied to accommodate mechanical or motorized lift systems that caregivers utilize to place or extract a user in or from a seating system.

The seating system in accordance with the disclosed subject matter may additionally or alternatively include other methods for adjusting the operation of the seating system based on sensor readings. For example, the seating system may also include a heating device. The processor may activate the heating device when a temperature sensor reading falls below a certain threshold to provide warmth to the user, or at the request of the user. In some embodiments, the seating system may also include a cooling unit which may be activated by the processor when a temperature sensor reading exceeds a certain threshold, or at the request of the user. In some examples, the heating and/or cooling devices can be manually overridden and turned on or off by the user or the caregiver. The heating and cooling elements may have associated timers to limit the application of heating and cooling systems. The seating system may also be configured to deactivate the heating system, cooling system, and other systems of the seating system when the seating system detects that the user is no longer in the seating system, e.g., when a weight detected by a weight sensor drops below a seated weight threshold and/or when a pressure detected by the pressure sensor drops below a seated pressure threshold.

In some examples, the heating and/or cooling device may be integrated with a cushion. For example, the seating system may include heating elements to warm the air that is located in or is to be injected into the inflatable portions. In other examples, the seating system may include cooling elements (such as a heat exchange) to cool the air. A thermal exchange device may be stored on the underside of the seating system (e.g., in a chassis under the seat of a wheelchair).

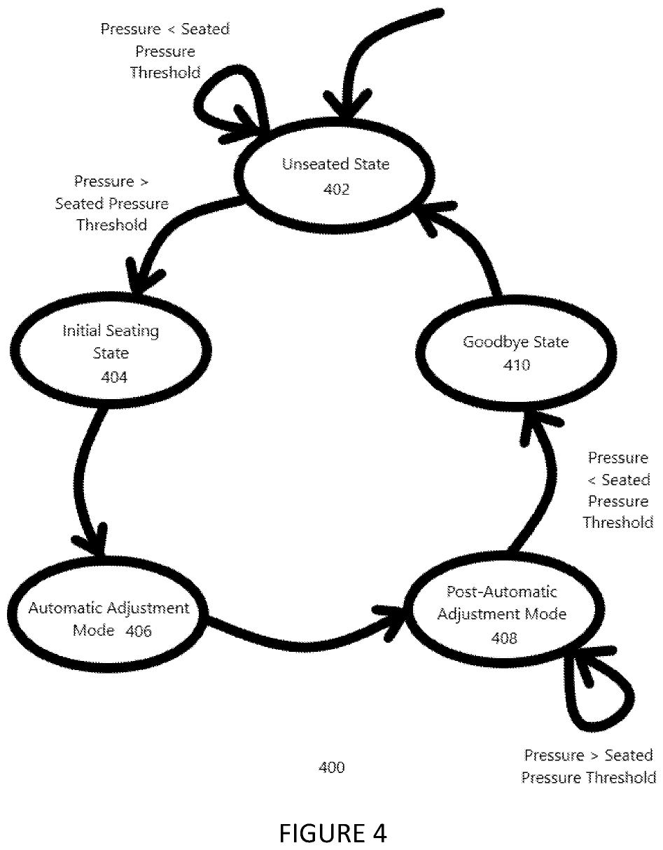

FIG. 4 is a state diagram 400 for adjusting the pressure of a cushion on a seating system in accordance with the disclosed subject matter.

The first state 402 may be an unseated state corresponding to a time period before the user sits in the seating system. For example, the seating system may enter the first state 402 as soon as the seating system is turned on. The seating system may remain in the first state 402 as long as one or more thresholds is not exceeded. For example, in some embodiments the seating system may remain in the first state until the pressure measurements from a plurality of pressure sensors on a cushion meet or exceed a seated pressure threshold value. In some other embodiments, the seating system may remain in the first state until the weight measurements associated with a weight sensor meet or exceed a seated weight value.

When the user sits in the seating system, the seating system will transition from the first state 402 to a second state 404. This transition may occur when the pressure measurements and/or weight measurements meet or exceed a threshold value as discussed above. In some examples, when the user enters the second state 404, the seating system may invite the user to adjust the cushion. For example, the seating system may display an automatic adjustment button on a display screen of the seating system and/or may cause a software application on a computing device to send the user and/or the caregiver an alert inviting them to enter the software application and start the adjustment process.

The seating system may then transition from the second state 404 to a third state 406. In some embodiments, the seating system may automatically transition from the second state 404 to the third state 406 after a set period of time, e.g., thirty seconds after the seating system enters the second state 404. In some other embodiments, the seating system may transition from the second state 404 to the third state 406 based on user input, e.g., when the user and/or caregiver selects the automatic adjustment button on the display screen of the seating system or through the software application.

Upon entering the third state 406, the seating system will automatically adjust the pressure of one or more inflatable chambers in the cushion. In some examples, the seating system may adjust the pressure of one or more inflatable chambers based on a profile specifying a pressure value for each user. In some embodiments, the seating system may adjust the pressure of one or more inflatable chambers based on a recommended profile specifying recommended pressure values for each user. In some examples, the recommended profile may be determined based on user information such as height, weight, and body measurements. In some other examples, the recommended profile may be determined based on historical pressure data.

After completing the automatic adjustment of the pressure for one or more inflatable chambers, the seating system may enter a fourth state 408. In the fourth state 408, the seating system may continue adjusting the pressure values for one or more inflatable chambers based on, e.g., pressure readings and user input. In some examples, the seating system may automatically adjust the pressure of the cushion as described above with reference to FIG. 2 when in the fourth state 408. In some examples, the seating system may adjust the pressure of the cushion based on user input as described above with reference to FIG. 3 when in the fourth state 408. In some examples, the seating system may adjust the pressure based on learned preferences, historical pressure data, and/or user behavior when in the fourth state 408.

The seating system may remain in the fourth state 408 as long as the weight and/or pressure measurements meet or exceed seated threshold values. When the weight and/or pressure measurements drop below the seated threshold values, the seating system may transition from the fourth state 408 to the fifth state 410. In some examples, the seating system will remain in the fourth state 408 until pressure measurements from all of a plurality of pressure sensors drop below the seated threshold value. In some examples, the seating system may remain in the fourth state 408 for a period of time after the weight and/or pressure measurements drop below the seated threshold to avoid moving to the fifth state 410 when the user is only briefly out of the seating system (e.g., when the user is repositioning or being repositioned). In some examples, the seating system may store pressure sensor data for a session when the seating system enters the fifth state 410. In some other examples, the seating system and/or software application may issue a goodbye message when the seating system enters the fifth state 410. The seating system may then transition from the fifth state 410 back to the first state 402 to await the user's return.

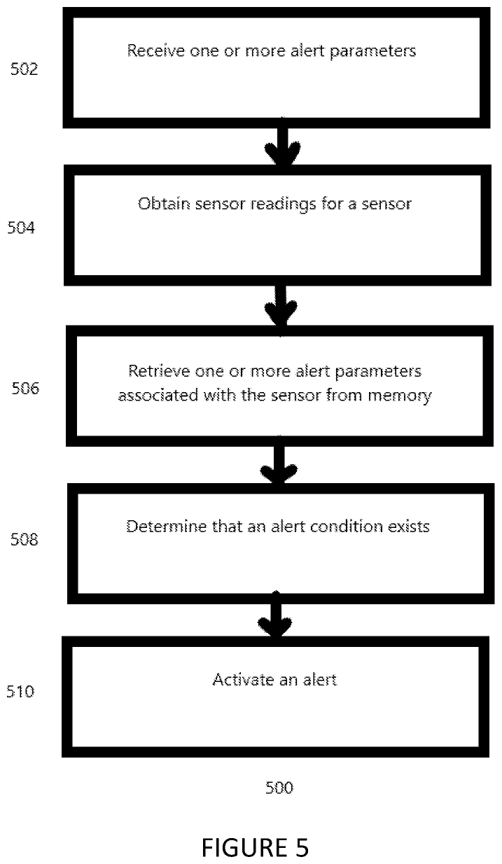

In some embodiments of the disclosed subject matter, the seating system may be configured to monitor the health of the user and/or the operation of the seating system, and to provide an alert when an unsafe condition is detected. FIG. 5 is a flowchart 500 describing a method for activating an alert in accordance with embodiments of the disclosed subject matter.

One or more alert parameters may be specified at 502. The alert parameters may be specific to a sensor. In some examples, the alert parameters may include a maximum value. For example, a maximum value for a moisture detector may be set such that any amount of moisture exceeding that value will trigger an alert. In some examples, the alert parameters may include a minimum value such as a minimum blood glucose level such that a blood glucose reading below that amount will trigger an alert. In some other examples, the alert parameters may specify a range including a lower limit and an upper limit. The range may specify an acceptable range for the sensor readings such that any value outside of that range will trigger an alert. For example, the alert parameters may identify an acceptable blood oxygen range, an acceptable heart rate range, or an acceptable temperature range.

In some examples, alert parameters may be set for two or more levels of alerts. For example, the alert parameters for a blood glucose sensor may identify a first blood glucose minimum associated with a first level alert and a second blood glucose minimum associated with a second level alert.

In some examples, alert parameters may be set based on input from the medical provider. For example, the medical provider may provide alert parameters corresponding to a user's medical condition.

The system may then obtain sensor readings for a sensor at 504. The sensor may be, for example, blood oxygen sensor such as a pulse oximeter, a heart rate sensor, a temperature sensor, a blood glucose sensor, a blood pressure sensor, a weight sensor, a speed sensor, a pressure sensor, an orientation sensor, or other sensors as known in the art. One or more processors (such as the one or more processors 104 as described in connection with FIG. 1) may receive the sensor readings from the sensor through wired or wireless communication.

The processors may then retrieve the one or more alert parameters associated with the sensor from memory at 506. The one or more alert conditions may be stored in internal memory 106a or external memory 106b as described above in connection with FIG. 1.

The processors may then determine whether an alert condition exists at 508. The processors may determine whether an alert condition exists based on the sensor reading and the retrieved alert parameters. For example, if the alert parameters identify an acceptable range, the processor may determine that an alert condition exists when the sensor reading is outside of the acceptable range.

If the processors determines that an alert condition exists, the processor activates an alert at 510. In some embodiments, the processors activate a visual indicator when an alert condition exists. For example, the processors may illuminate a warning light when an alert condition exists. In some other examples, the processors may change the color of a warning light (e.g., from green to red) when an alert condition exists. In some embodiments, the processor may activate an audio alert such as providing a voice message or other audio indication through a speaker. In some other examples, the processor may cause a text message to be sent to a local or remote display device concerning the alert. In some examples, the text message may include details about the alert including the type of alert (e.g., high heart rate or low heart rate) and the sensor reading (e.g., 60 bpm). In some examples, the text message may also provide suggested actions (e.g., an instruction to call the user's medical provider).

In some examples, the processors may transmit a message to an electronic medical records system and/or a software application associated with the user's medical provider. In some examples, the processors may transmit the message to the medical provider only when parameters set by the medical provider are satisfied. In some examples, the processors may transmit a text message to one or more cell phones specified by a user, such as a cell phone associated with the caregiver, a cell phone associated with a medical provider, or a cell phone associated with a family member such as the user's parent, spouse, or child.

Additional information about the alert may be provided based on a request from the user, caregiver, medical provider, or other interested party. For example, upon noticing the alert, a person may send a request for additional information about the alert to the seating system (e.g., through an application on the person's mobile device or a display screen integrated with the seating system). In some examples, the person may also send a request for an updated sensor reading (e.g., because the user or caregiver has adjusted the sensor and wants to check whether the initial sensor reading was correct). The seating system may receive the request, send a response including details about the alert, request an updated sensor reading from the sensor, and send a response including the updated sensor data.

In some examples, the processor may adjust the operation of the seating system if an alert condition is detected. For example, if the processor determines that the user's temperature is above a threshold value, the processor may activate the cooling system to provide relief to the user.

The processor may also store the sensor readings. In some examples, the processor may store the sensor readings by writing the data to memory. In some examples, the processor may store the sensor readings only when an alert condition is detected.

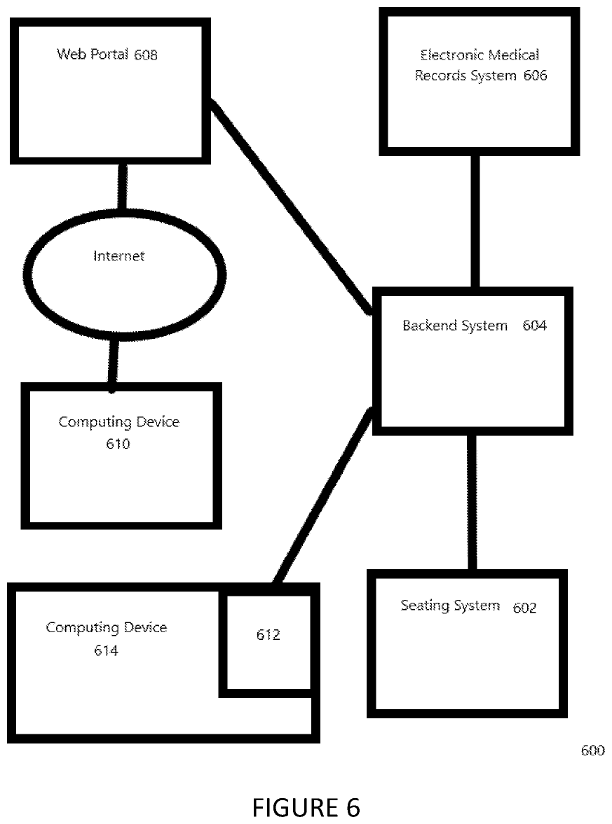

FIG. 6 is a block diagram of a networked system 600 in accordance with embodiments of the disclosed subject matter. The system 600 may include a seating system 602, a backend system 604, and an electronic medical records system 606. The seating system 602 may be, for example, a wheelchair.

The seating system 602 may communicate with the backend system 604 through one or more communication units or communication ports. The backend system 604 may include one or more transceivers that allow the backend system 604 to receive information from and send information to the seating system 602. The backend system 604 may also include one or more memories configured to store data received from the seating system 602. For example, the backend system 604 may include one or more computers and/or one or more servers. In some examples, the one or more servers may include one or more cloud servers. In some examples, the backend system 604 may be a distributed system.

The seating system 602 may include one or more sensors. In some examples, the sensors may be biometric sensors. For example, the one or more sensors may include a heart rate sensor, a blood pressure sensor, a blood glucose sensor, a blood oxygen sensor such as a pulse oximeter, a temperature sensor, a pressure sensor, a moisture sensor, or any other sensors as known in the art. The seating system 602 may be programmed to transmit sensor data from any of the sensors to the backend system 604 for storage. In some embodiments, the seating system 602 may be configured to transmit the sensor data to the backend system 604 in real-time. In some examples, the seating system 602 may be programmed to transmit the sensor data to the backend system 604 at periodic intervals. In further embodiments, the seating system 602 may be configured to transmit the sensor data to the backend system 604 upon the occurrence of a particular event. For example, the seating system 602 may be configured to transmit sensor data to the remote data store if an alert condition is detected (e.g., the user's blood oxygen levels are outside of an acceptable blood oxygen range). As another example, the seating system 602 may be configured to transmit sensor data to backend system 604 in response to a request, e.g., from a medical provider or the backend system 604. As a further example, the seating system 602 may be configured to transmit sensor data when the seating system 602 is coupled to an electrical source (e.g., when the seating system 602 is plugged in at the end of the day) to conserve power (which may be desirable where only safe conditions are detected). Such a configuration may be particularly desirable where the data connection is wired. In some examples, the seating system 602 may be configured to transmit sensor data upon detecting that a communication channel has been re-established if, for example, the seating system 602 is unable to communicate with the backend system 604 for a period of time. In some embodiments, the user, caregiver, or medical provider may be offered the option of selecting how the seating system 602 will communicate the sensor data to the backend system 604. The transmission of data to the backend system 604 store may be wired or wireless.

In some examples, sensor data may be communicated and stored in an encrypted format. For example, the seating system 602 may include an encryption unit configured to encrypt the sensor data before it is transmitted to the backend system 604. The seating system 602 may also include a decryption unit configured to decrypt sensor data received from the backend system 604 (e.g., historical sensor data that may be requested by the user or caregiver). The backend system 604 may include similar encryption and decryption units. In embodiments of the disclosed subject matter, the sensor data may be stored in a format that is compliant with applicable regulations covering the protection of personal information such as, for example, regulations associated with HIPAA (the Health Insurance Portability and Accountability Act) in the United States and/or GDPR (General Data Protection Regulation) in Europe. In some examples, non-sensitive information may be transmitted and stored without such encryption and formatting precautions. For example, information about the speed and orientation of the seating system may not require the same protection as information about the user's heart rate and blood oxygen levels.

In some examples, the backend system 604 may also store additional information associated with the user. For example, the backend system 604 may store an account identifier associated with the user. In some other examples, the backend system 604 may store an identification of the user's medical provider or other persons who are authorized to view the user's information (e.g., the sensor data). In some examples, the backend system 604 may also include an identification of the types of data each authorized user may view. For example, while a user's medical provider may be granted access to all of the user's sensor data, the user may want a family member to have access to only limited information (e.g., the GPS location of the seating system, the user's heart rate, and/or the user's blood glucose level). The backend system 604 may also store account information for each authorized user, which may include a user identifier and a password.

In some examples, the backend system 604 may be configured to communicate with one or more electronic medical records systems 806. The electronic medical records system 606 may be an electronic medical records system used by the user's medical provider. In some examples, the electronic medical records system 606 may be an electronic medical records system used by another medical provider such as a hospital that is treating the user in an emergency situation. The backend system 604 may authenticate the electronic medical records system 606 using techniques as known in the art. By accessing the sensor data through the electronic medical records system 606, a treating medical provider may be able to better treat the user (e.g., by understanding the user's medical condition during a period before, during, and after an emergency medical event such as a heart attack or stroke).

In some examples, the electronic medical records system 606 may also transmit information to the backend system 604 and/or the seating system 602. For example, the user's medical provider may enter one or more alert parameters into the electronic medical records system 606. The medical provider may select the alert parameters based at least in part on the sensor data and/or the user's medical history. The electronic medical records system 606 may transmit the alert parameters to the backend system 604, which may store the alert parameters in memory and transmit the alert parameters to the seating system 602. In some other embodiments of the disclosed subject matter, the electronic medical records system 606 may be configured to communicate directly with the seating system 602. In some other examples, the electronic medical records system 606 may transmit a treatment plan for the user. The medical provider may develop the treatment plan based at least in part on the sensor data received from the seating system 602 or the backend system 604, including the historical sensor data stored at the backend system 604. The treatment plan may be made available to the user at the seating system 602 (e.g., via a display screen of the seating system 602) or via a computing device including a software application for accessing the treatment plan.

In some examples, the backend system 604 may make sensor data available to the user, the caregiver, the medical provider, or other persons designated by the user (e.g., non-caregiver family members). In some examples, the information may be accessible through existing electronic medical record channels as discussed above.

In some examples, the information may be available through the Internet. For example, the system 600 may include a web portal 608 coupled to the backend system 604. The user, caregiver, medical provider, or others may access a website associated with the web portal 608 by entering the appropriate address (i.e., URL) on a computing device 610 connected to the Internet. The web portal 608 may send a request for a user identifier and password to the computing device 610 (e.g., as part of the HTML, or other code for the website). The web portal 608 may provide sensor data to the computing device 610 only if the user identifier and password match the user identifier and password of a user authorized to view the sensor data.

In some examples, the information may be available through a software application such as an application 612 running on a computing device 614. The software application 612 may be, for example, a desktop application or a mobile application. The computing device 614 may be, for example, a mobile phone or tablet device. The user, caregiver, medical provider, or other person may need to input a user identifier and password to access the user's sensor data through the software application 612.

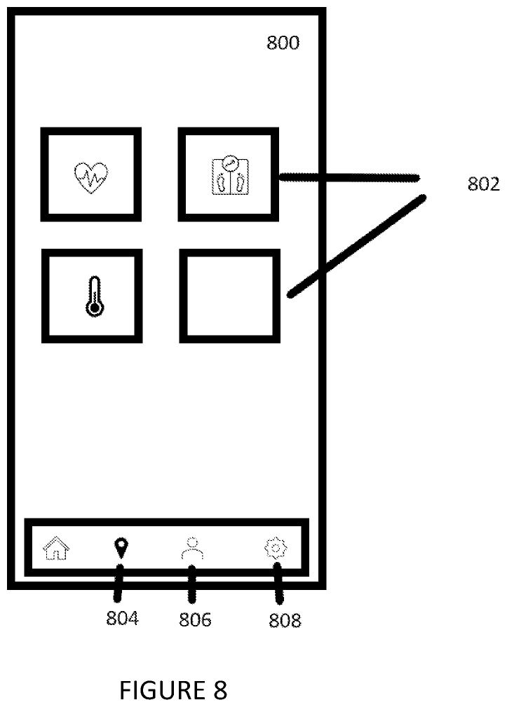

In some examples, one or more persons designated by the user may be provided with or given the option of receiving reports related to the user's sensor data, such as periodic (e.g., daily or weekly) reports and/or alerts, through the web portal 608 or the software application 612. For example, the backend system 604 may generate a report based on the sensor data received from the seating system 602. The report may be generated based on the sensor data for a predetermined time period, such as the last day or the last seven days. In some examples, each sensor measurement may be tagged with a time indicator that indicates when the measurement was taken. The backend system 604 may transmit the report to the electronic medical records system 606 associated with the user's medical provider. The backend system 604 may also make the report available through the web portal 608 and the software application 612, e.g., in response to a request to access the report. Similarly, an alert may be sent to the electronic medical records system 606, via the webs portal 608, and/or through the software application 612 when the user's sensor data indicates an alert condition, as discussed in greater detail in connection with FIG. 8 above.

In some examples, the seating system may be controlled based on commands provided by a caregiver or medical provider, e.g., through the software application 612. For example, the caregiver may use the mobile application 612 to cause the seating system to stop moving (e.g., by shutting off a motor or other propulsion system, or applying an electronic brake). As another example, a medical provider may request one or more sensors to perform another reading or perform self-diagnostics to double-check a reading or confirm that the sensor is properly operating. The medical provider's request may be transmitted to the seating system 602 via the electronic medical records system 606, the web portal 608, or the software application 612.

In some examples, the user, caregiver, or medical provider may configure the seating system through the software application 612 or by using another computing device (e.g., computing device 610).

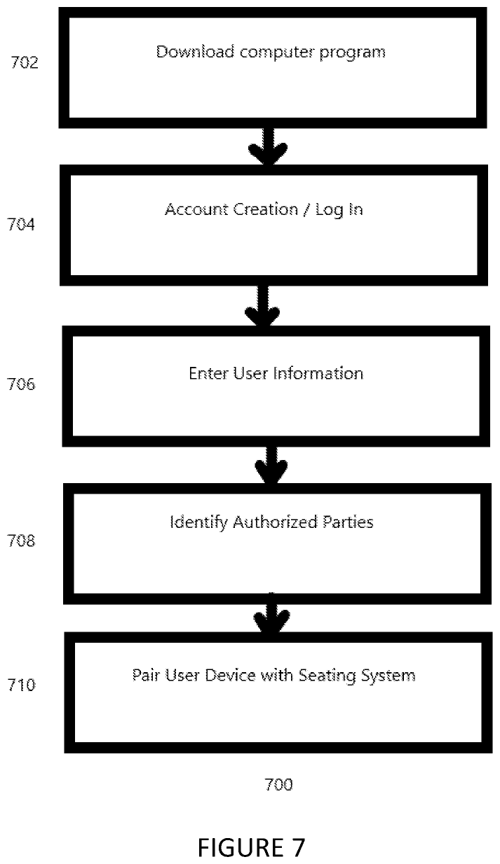

FIG. 7 is a flowchart 700 showing the steps for setting up a seating system in accordance with some embodiments of the disclosed subject matter. The user or a caregiver, family member, or other person may download a computer program (e.g., a mobile application) to a user device such as a mobile phone or tablet device at 702. In some examples, the person may scan a QR code provided with the seating system to download a mobile application. In some other examples, the person may download a mobile application through an application store. In some other examples, the person may download a desktop application over the Internet. In embodiments where the seating system is set up through a website, the person may not need to download any software. In some other examples, the computer program may be pre-stored in a memory of the seating system and the person may access the computer program through, for example, a display screen on the seating system.

The person may then create a new account or log into an existing account at 904. The person may create a new user identifier and password when setting up the account or may enter an existing user identifier and password for the existing account.

The person may then enter user information at 706. The user information may include basic information such as name, age, gender, and the like. In some examples, the person may identify one or more medical conditions or clinical diagnoses associated with the user. For example, the person may be allowed to identify one or more conditions from the following exemplary list: paraplegic, broken limb, quadriplegic, muscular dystrophy, disorientation, and/or neurological. In some other examples, the person may be prompted to indicate the medical conditions or clinical diagnoses, e.g., by entering established diagnostic codes. In some examples, the medical condition information indicated by the person may be used for operation of the wheelchair, e.g., to determine adjustments to the inflation of the cushion and/or to determine emergency conditions associated with one or more of the sensors integrated with the seating system. In some examples, the person may identify additional user information such as age or birthdate, height, and/or weight. In some examples, the user information may be used to provide recommended settings or automatically configure the settings of the seating system. In some examples, the person may also identify a user activity level. The identified activity level may be used in operation of the seating system, e.g., in determining how to adjust the inflation of the cushion. In some examples, the person or another person may subsequently modify information previously entered by the person.

The person may also identify one or more caregivers or other persons who may be given access to information from the seating system at 708. For example, the person may enter a phone number or email address associated with a caregiver to be given access to the seating system, which may cause a backend system (such as backend system 604 as described with reference to FIG. 6) to send an email, text message, or other communication to the caregiver inviting them to create an account and/or accept the invitation to access information about the user's seating system. In some examples, the person may also identify a level of access for each identified caregiver. For example, the person may allow a family member that does not provide caregiving functions to view sensor data from the seating system but not control the operation of the seating system. In some examples, the person may allow the family member access to only certain types of sensor data. The person may allow a caregiver to view the sensor data and control the operation of the seating system (e.g., to inflate or deflate the cushion). In some examples, the person may select one or more persons who are allowed to view the location of the seating system but not to view any of the sensor data or control the operation of the seating system, which may be helpful if the user cannot be located. The person may also identify one or more medical or other care providers, who by default may be given access to all sensor data from the seating system to assist in monitoring the patient's health. The person may change the default settings to limit the medical or other care provider's access to data from the seating system.

The person's device may then pair with the seating system via wireless communication at 710. For example, a person's device may include a transceiver such as a Bluetooth transceiver that may communicate with a corresponding transceiver of the seating system. In some examples, the person may need to enter some information about the seating system (e.g., an identification number printed on the seating system) to pair the seating system with the device. The seating system may thereafter send information to the person's device or another paired device (such as a caregiver's device, which may be paired in a similar manner).

In some examples, the user, the user's medical provider, and the user's authorized caregivers or other persons may also be permitted to access information from the seating system through a computer program or website that communicates with the backend system. In such embodiments, no pairing may be required.

FIG. 8 shows a screenshot of an interface of a software application through which a user, caregiver, medical provider, or other person may view data from the sensors on a seating system in accordance with the disclosed subject matter. as the software application may be a software application 612 as described with reference to FIG. 6. The software application runs on a computing device 800 and may be viewed, e.g., on a display screen that is part of the seating system or a display of a person's mobile device such as a phone or tablet. In some examples, the software application may request and/or receive current and/or historical sensor data associated with the seating system directly from the seating system or via a backend system such as backend system 604 as described with reference to FIG. 6.

The interface may display a screen showing providing display information 802 about one or more sensors on the seating device. In some examples, the display information 802 may be current sensor readings In some examples, the display information 802 may be arranged as an array of one or more icons or tiles. The current sensor readings may include a current heart rate, a current weight, a current temperature, and a current charge for the seating device. The current sensor readings may also include current pressure sensor readings. The current sensor readings may also include current readings from any of the other sensors of a seating system as described herein. The current sensor readings may be received via a direct connection (e.g., a wireless connection) between the seating system and the computing device on which the software application is running. In some other examples, the current sensor readings may be transmitted from the seating system to the computing device via a backend system. The current sensor readings may be displayed in an alphanumeric format, a graphical format, or both. In some examples, the user can request additional information about the sensor readings. For example, the display information 802 may show alphanumeric information about the current sensor readings and the user may access for detailed information (e.g., a pressure maps associated with pressure sensors) by selecting an icon or tile associated with the sensor. In some examples, the sensor readings available to the person may depend on permissions set by the user. For example, a medical provider may have access to all sensor readings while a person such as the user's spouse or child may have access to limited or no sensor readings.

In some examples, the display information 802 may also include historical sensor readings. In some examples, the historical sensor readings may include historical sensor readings for one or more intervals, such as sensor readings for the past day, sensor readings for the past week, and/or sensor readings for the past month. In some examples, the historical sensor readings may be accessible by a user selecting a sensor. For example, the user may be able to access historical sensor readings for a weight sensor by selecting an icon or tile showing the current sensor reading for the weight sensor. In some examples, the seating system may not store historical data readings for the full time period. The software application may therefore need to retrieve historical sensor readings from a backend system even if the software application is in direct contact with the seating system. The data may be displayed in an alphanumeric format, in a graphical format, or both.

In some examples, the user also may be able to access location information for the seating system through the software application. For example, the user may be able to access location information by selecting an icon 804 associated with the location information.

In some examples, the user may also be able to access user profile information and settings for the seating system through the software application. For example, the user may access the user profile by selecting the icon 806 associated with the user profile. The user may be able to add or modify user information, caregiver information, or other information as discussed with reference to FIG. 7. As another example, the user may access settings for the seating system by selecting the icon 808 associated with the settings. The user may be able to modify operational parameters associated with the seating system through the software application. For example, the user may be able to set a minimum temperature and a maximum temperate. This information may be transmitted to the seating system. If the temperature measured at the seating system drops below the minimum temperature, the seating system may activate a heating device to bring the temperature back to the minimum temperate. Similarly, if the temperature measured at the seating system drops below the maximum temperature, the seating system may activate a cooling device to bring the temperature back to a maximum temperature.

In some examples, other information may be available through the software application. For example, the user and/or caregiver may be able to access a treatment plan for the user through the software application.

The information available through the software application as discussed with reference to FIG. 8 may alternatively be accessed through a website (via a web portal such as web portal 608 as discussed with reference to FIG. 6) or at the seating system (e.g., at a display screen attached to the seating system).

In some examples, the seating system or a software application (e.g., the user's or caregiver's mobile application) may be configured to communicate with a medical provider computer system (which may be an electronic medical record system such as electronic medical records system 606 as described with reference to FIG. 6). In some examples, the seating system or software application may communicate with the medical provider computer system to facilitate the provision of medical care to the user.

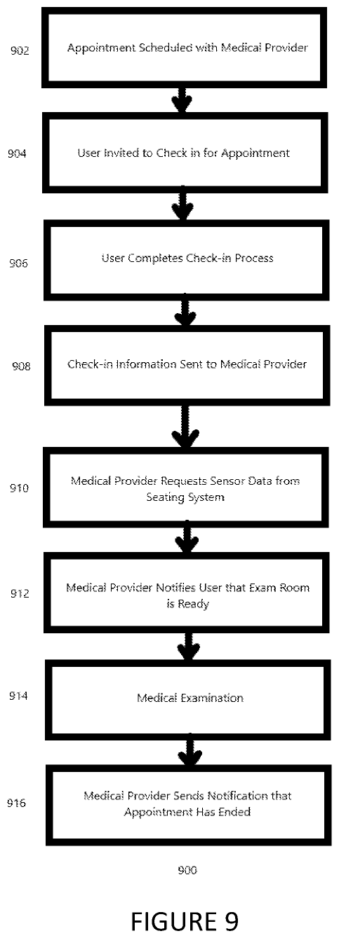

FIG. 9 is a flowchart describing a method for communicating with a medical or other care provider computer system in accordance with some embodiments of the disclosed subject matter.

The user may schedule an appointment with a medical or other care provider at 902. In some examples, the user may schedule the appointment through a software application such as software application 612 as described with reference to FIG. 6. In some examples, the user may schedule the appointment through the seating system, e.g., using a display screen attached to the seating system. In some examples, the user may schedule the appointment through other communications channels, e.g., in-person (such as at the end of a previous appointment) or via telephone. The medical provider computing device may send a confirmation to the software application. For example, an electronic medical record system (such as electronic medical records system 606) may transmit a confirmation to a software application (such as software application 612), either directly or via a backend system (such as backend system 604). The appointment information may be stored in the device on which the software application is running, in a memory of the seating system, or on a backend system. The software application, seating system, or backend system may be configured to provide reminders to the user and/or caregiver as the appointment approaches.

The user may be invited to check in for the appointment at 904. In some examples, the seating system or software application on a computing device may detect that the user is approaching the office of the medical provider, e.g., using a location tracker such as a GPS system in the seating system or user device, and may send a notification to the user inviting the user to check in for the appointment. For example, the notification may be provided to the user when the seating system or user device is determined to be within half a mile of the medical provider office. In some examples, the notification may be provided only if the current time is within a certain amount of time before the scheduled appointment, e.g., half an hour.

The user may then complete the check-in process at 906. In some examples, the user may complete the check-in process through the user device or seating system, which may minimize the amount of time the user spends in a waiting room with other patients that may be ill, which in turn may minimize the risk that the user becomes ill when visiting the medical provider office.

The check-in information may then be transmitted to the medical provider computer system at 908. The check-in information may be transmitted to the medical provider directly from the user device or seating system or indirectly, e.g., through the backend system. The provider medical office may use that information to complete the registration process for the patient.

As part of the registration process, the medical provider computer system may request the sensor data from the seating system at 910. In some examples, the medical provider computer system may request historical sensor data stored at the backend system and/or in a memory of the seating system. In some other examples, the medical provider computer system may additionally or alternatively request current sensor data from the seating system. The sensor data may be provided to the medical provider for use during the user's appointment.

The medical provider computer system may notify the medical provider is ready to begin the appointment at 912. In some examples, the medical provider system may determine when an exam room is available for the user. For example, the medical provider computer system may receive input from a member of the medical provider's office indicating that an exam room is available. The medical provider computing system may transmit a notification to the user device and/or seating system indicating that the exam room is available, further limiting the amount of time the user may be required to wait in a waiting room.

The user may thereafter enter the exam room for the appointment at 914. A medical provider (e.g., a doctor or nurse) may examine the user and review sensor data in determining the appropriate course of treatment. In some cases, the medical or other care provider (such as a seating specialist, physical therapist, or occupational therapist) may request additional sensor data via the medical provider computer system, control the seating system (e.g., adjust the inflation of the cushion) via the medical or other care provider computer system, or update alert parameters associated with one or more of the sensors of the seating system via the medical or other care provider computer system.

Following the appointment, the medical provider computer system may provide a notification that the appointment has ended to the seating system and/or user device at 916. In some examples, the medical provider computer system may also provide additional information (e.g., care recommendations) to the user through the user device. In some examples, the user may also be invited to schedule a next appointment with the medical provider through the seating system and/or mobile device, thereby further limiting the amount of time the user needs to spend in the waiting room or other common areas of the medical provider office.

In some examples, the medical provider computer system may communicate with a device associated with an authorized caregiver or other authorized person as an alternative or in addition to communicating with the seating system and/or user device.

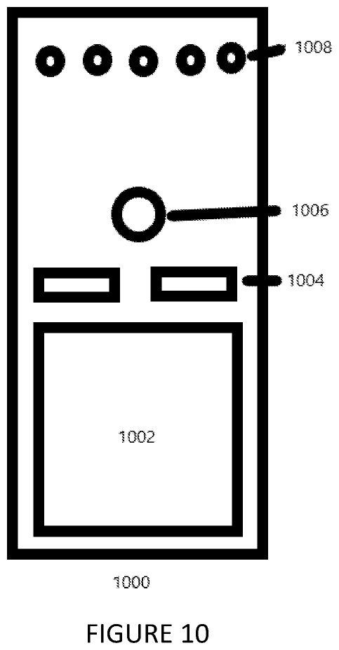

FIG. 10 is a block diagram of an armrest 1000 of a seating system in accordance with embodiments of the disclosed subject matter.

The armrest 1000 may include a display screen 1002. In some other examples, the display screen 1002 may be located in other locations on the seating system such as behind the back portion so that it is viewable to the caregiver rather than the user or offset from the armrest and viewable by the user and other persons. The display screen 1002 may be configured to display information for the user. In some examples, the display screen 1002 may have a touchscreen and may also be configured to receive input from the user. The display screen 1002 may display any information discussed herein to the user. For example, in some examples the display screen 1002 may display sensor measurements. In some examples, the display screen 1002 may display text alerts when an alert condition is detected. In some examples, the display screen 1002 may display an icon requesting the user to begin an automatic cushion adjustment. In some examples, the display screen 1002 may display an icon for the user to select when they are uncomfortable and would like to request a seat adjustment.

In some examples, the display screen 1002 may display instructions for a user. In some examples, the instructions may be based on a medical condition associated with the user. For example, if the user is a diabetic, they may be required to take blood glucose measurements on a periodic basis. The seating system may be configured to display a reminder on the display screen 1002 when it is time for the user to measure their blood sugar. In some other examples, the seating system may be configured to display a reminder for the user to take medicine according to a medicine schedule on the display screen 1002.

The armrest 1000 may also include one or more communication interfaces 1004. The communication interfaces 1004 may be USB ports. In some examples, the seating system may include one or more sensors that are removably attachable to the seating system via communication interfaces 1004. The one or more removable sensors may be stored in a storage location (such as a, flexible pack, box, or drawer) located underneath the seat portion of the seating system or behind the seat back when not in use. The one or more removable sensors may be, for example, a pulse oximeter configured to transmit blood oxygen readings to the memory of the seating system via the communication interfaces. In some other examples, the communication interfaces 1004 may be located on other places on the seating system, such as on the side of the armrest 1000 or on a front or side of the seat portion.

The armrest 1000 may include one or more input buttons 1006. In some examples, the input buttons 1006 may be a general button, e.g., it may be pushed to activate a variety of functions. For example, the armrest 1000 may include a first input button 1006 and a second input button 1006 that may be used to respond yes and no, respectively, to a question presented to the user (e.g., through the display screen 1002). In some other examples, each of the one or more input buttons may be specialized. For example, a first input button 1006 may be used only to request adjustments to the pressure of the cushion of the seating system, a second input button 1006 may be used to adjust the incline of the seat portion of the seating system, and a third input button 1006 may be used to activate the massage functionality of the cushion.

The armrest 1000 may include one or more indicators 1008, which may be indicator lights. For example, a first indicator light 1008 could correspond to the power state of the seating system. The first indicator light 1008 may be illuminated in green when the seating system is charged above a threshold charge (e.g., 20% battery) and may be illuminated in red when the system charge drops below the threshold charge. In some other examples, one or more of the indicator lights 1008 may indicate an alert condition exists when illuminated. For example, one or more of the indicator lights 1008 may be illuminated when the seating system measures a moisture level above a moisture threshold, or a heart rate above a heart rate threshold.

In some examples, the armrest 1000 may include additional components such as a speaker, a microphone, a joystick, or one or more integrated sensors such as a heart rate sensor or a temperature sensor. In some examples, the armrest 1000 may also include convenience components such as a cupholder.

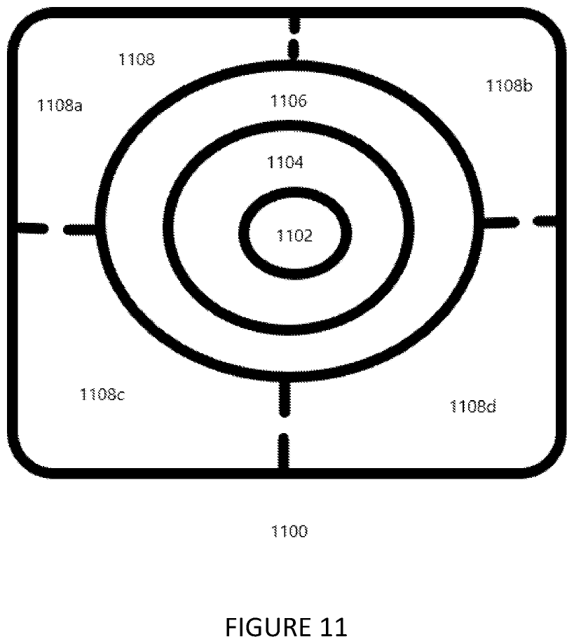

FIG. 11 is a depiction of a cushion 1100 including multiple inflatable portions in accordance with some embodiments of the disclosed subject matter.

Cushion 1100 may include a plurality of inflatable chambers. For example, cushion 1100 may include a plurality of inflatable chambers formed as concentric circles or ovals, such as inflatable chambers 1102, 1104, and 1106. Cushion 1100 may also include one or more chambers of a different size or shape, such as inflatable chamber 1108. Each of the inflatable chambers 1102, 1104, 1106, and 1108 shown in FIG. 11 may be divided into two or more inflatable chambers. For examples, the cushion 1100 may include inflatable chambers 1108a, 1108b, 1108c, and 1108d as indicated by the dashed lines. Each of the inflatable chambers may include one or more chamber pressure sensors located inside or outside the chamber or at an opening thereof. In some other examples, the cushion 1100 may include a plurality of square, rectangular, circular, or other regularly shaped chambers. In some examples, the cushion may include more or smaller cushions around areas where finer adjustments may be needed, such as at the back and toward the middle of the cushion 1100.

The cushion 1100 may also include one or more surface pressure sensors located on the surface of the cushion. In some examples, the cushion 1100 may include one or more surface pressure sensors located above each inflatable chamber. In some examples, the surface pressure sensors may be located at regular distance intervals (e.g., every square inch) on the cushion 1100. In some other examples, the surface pressure sensors may be clustered around locations where a user is likely to experience pressure or pain. For example, the cushion 1100 may have more surface pressure sensors located near the middle and the back of the cushion 1100.

The disclosed subject matter includes, but is not limited to, the following embodiments, and variations of these that would occur to those of skill in the art upon review of the present disclosure:

Embodiment 1. A human transport system comprising:

one or more processors;

one or more sensors;

one or more memory units for storing readings from the sensors; and

one or more communication systems for transmitting the readings from the sensors to a remote computer system.

Embodiment 2. The human transport system of Embodiment 1, wherein the human transport system is a wheelchair.

Embodiment 3. The human transport system of Embodiments 1 or 2, wherein the one or more sensors includes at least one of a weight sensor, a temperature sensor, a pressure sensor, and a blood oxygen sensor.

Embodiment 4. The human transport system of any of Embodiments 1 through 3, wherein the one or more communication systems include at least one of a Bluetooth communication system and a Wi-Fi communication system.

Embodiment 5. The human transport system of any of Embodiments 1 through 4, further including a location tracker.

Embodiment 6. The human transport system of any of Embodiments 1 through 6, further including a sensor for measuring the speed of the patient transport system.

Embodiment 7. The human transport system of any of Embodiments 1 through 6, further including one or more indicators.

Embodiment 8. The human transport system of Embodiment 7, wherein the processor is configured to active at least one of the one or more indicators upon detecting an alert condition.

Embodiment 9. The human transport system of Embodiment 8, wherein the processor displays a message on a display screen when the unsafe condition is detected.

Embodiment 10. The human transport system of any of Embodiments 1 through 9, further including one or more armrests.

Embodiment 11. The human transport system of Embodiment 10, wherein one or more indicator lights associated with the one or more sensors are located on the one or more armrests.

Embodiment 12. The human transport system of Embodiment 11, wherein the indicator lights include a first state associated with a safe condition and a second state associated with an emergency condition.

Embodiment 13. The human transport system of Embodiment 13, wherein the indicator lights include a third state associated with a warning condition.

Embodiment 14. The human transport system of Embodiment 14, wherein each state is associated with a range of values for the corresponding sensor.

Embodiment 15. The human transport system of any of Embodiments 1 through 14, further including a cushion.

Embodiment 16. The human transport system of Embodiment 15, wherein the cushion includes a plurality of inflatable chambers.

Embodiment 17. The human transport system of Embodiment 17, wherein a processor is configured to selectively inflate or deflate the plurality of inflatable chambers.

Embodiment 18. The human transport system of Embodiment 17, wherein the processor is configured to selectively inflate or deflate the plurality of inflatable chambers based on instructions stored in memory.

Embodiment 19. The human transport system of Embodiments 17 or 18, wherein the processor is configured to selectively inflate or deflate based on user input.

Embodiment 20. The human transport system of Embodiments 16 through 19, wherein the cushion includes one or more surface pressure sensors.

Embodiment 21. The human transport system of Embodiment 20, wherein a processor is configured to selectively inflate or deflate chambers of the cushion based on pressure readings from the one or more pressure sensors.

Embodiment 22. The human transport system of Embodiments 16 through 21, wherein the cushion includes one or more in-chamber pressure sensors.

Embodiment 23. The human transport system of Embodiment 22, wherein a processor is configured to selectively inflate or deflate chambers of the cushion based on pressure readings from the one or more in-chamber sensors.

Embodiment 24. The human transport system of any of Embodiments 16 through 23, wherein the processors are configured to obtain pressure readings from one or more pressure sensors associated with a cushion, identify one or more inflatable chambers based on the pressure readings, and to adjust the pressure in the one or more inflatable chambers.

Embodiment 25. The human transport system of Embodiment 24, wherein the processors are configured to request the pressure readings based on user input.

Embodiment 26. The human transport system of Embodiments 24 or 25, wherein the one or more inflatable chambers are identified based at least in part on a medical condition associated with the user.

Embodiment 27. The human transport system of Embodiments 24 through 26, wherein the one or more inflatable chambers are identified based at least in part on a threshold pressure value.

Embodiment 28. The human transport system of Embodiments 24 through 27, wherein the one or more inflatable chambers are identified based at least in part on a pressure profile specifying pressure values for each of the plurality of inflatable chambers.

Embodiment 29. The human transport system of Embodiment 28, wherein the pressure profile comprises a profile stored in the one or more memories based on prior user experience and/or user input.

Embodiment 30. The human transport system of Embodiment 28, wherein the pressure profile comprises a recommended profile based on at least one of a user height, a user weight, user body measurements, a medical condition associated with the user, learned user preferences, and recorded user behavior.

Embodiment 31. The human transport system of Embodiments 1 through 30, where the processors are configured to receive one or more sensors readings from the one or more sensors, retrieve alert parameters from the one or more memories, determine that an alert condition exists based on the alert parameters, and activate an alert.

Embodiment 32. A system comprising the human transport system of any of Embodiments 1 through 31 and one or more remote computers configured to receive and store sensor data from the one or more sensors of the human transport system.

Embodiment 33. The system of Embodiment 33, further comprising an electronic medical records system configured to request sensor data from the one or more remote computers or the human transport system.

Embodiment 34. The system of Embodiments 32 and 33, wherein the one or more remote computers are configured to detect an alert condition based on the sensor data from the one or more sensors.

Embodiment 35. The system of Embodiment 34, wherein the one or more remote computers are configured to transmit an alert to at least one of the user, the caregiver, the medical provider, or another authorized person based on detecting the alert condition.

Embodiment 36. The system as described in any of Embodiments 32 through 34, further comprising a web portal.

Embodiment 37. The system of any of Embodiments 32 through 36, wherein the one or more remote computers are further configured to receive a request for access to the sensor data, determine whether the requestor is authorized to access the sensor data based on authorization data received from the user, and transmit the sensor data to the requestor when the requestor is determined to be an authorized party.

Embodiment 38. The system of any of Embodiments 1 through 37, further comprising controlling one or more features of the human transport device based on the sensor data.

Embodiment 39. The system of any of Embodiments 1 through 38, wherein the system is compliant with applicable regulations such as HIPAA and/or GDPR and/or wherein access to the sensor data through a requesting device is managed in compliance with such applicable regulations.

The foregoing merely illustrates the principles of the disclosed subject matter. Various modifications and alterations to the described embodiments will be apparent to those skilled in the art in view of the inventors' teachings herein. It will thus be appreciated that those skilled in the art will be able to devise numerous systems and methods which, although not explicitly shown or described herein, embody the principles of the disclosed subject matter and thus are within it spirit and scope. Such modifications and alterations are within the scope and spirit of the present invention, as set forth in the following claims. Further, the invention(s) described herein is capable of other embodiments and of being practiced or of being carried out in various ways. It is to be understood that the phraseology and terminology used herein is for the purpose of description and should not be regarded as limiting.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

The features of the various embodiments described herein are not intended to be mutually exclusive when the nature of those features does not require mutual exclusivity. Instead, features and aspects of one embodiment may be combined with features or aspects of another embodiment. Additionally, the description of a particular element with respect to one embodiment may apply to the use of that particular element in another embodiment, regardless of whether the description is repeated in connection with the use of the particular element in the other embodiment.

Examples provided herein are intended to be illustrative and non-limiting. Thus, any example or set of examples provided to illustrate one or more aspects of the present disclosure should not be considered to comprise the entire set of possible embodiments of the aspect in question. Examples may be identified by use of the terms or phrases “for example,” “such as,” “by way of example,” “e.g.,” and other language commonly understood to indicate that what follows is an example.

What is claimed is:

1. A method for controlling the pressure in a cushion comprising:
providing at least one cushion comprising a plurality of inflatable chambers;
obtaining pressure measurements from a plurality of pressure sensors associated with the at least one cushion;
identifying at least one inflatable chamber of the at least one cushion based at least in part on the pressure measurements; and
adjusting a pressure of the at least one inflatable chamber.

2. The method of claim 1, wherein the at least one cushion is positioned on a wheelchair.

3. The method of claim 1, wherein the plurality of pressure sensors comprise at least one of a surface pressure sensor and a chamber pressure sensor.

4. The method of claim 1, further comprising:
generating a pressure map based at least in part on the pressure measurements;
identifying one or more locations on the cushion based at least in part on the pressure map; and
identifying the at least one inflatable chamber based at least in part on the one or more locations and a seat map.

5. The method of claim 1, wherein identifying the at least one inflatable chamber comprises identifying the at least one inflatable chamber associated with a pressure measurement above a threshold value.

6. The method of claim 5, wherein adjusting the pressure of the at least one inflatable chamber comprises decreasing the pressure of the at least one inflatable chamber.

7. The method of claim 1, wherein identifying the at least one inflatable chamber comprises
comparing the pressure measurements with a profile comprising pressure values for each of the one or more inflatable chambers; and
identifying the at least one inflatable chamber where a pressure measurement from a chamber pressure sensor associated with the at least one chamber does not match a corresponding pressure value from the profile.

8. The method of claim 7, wherein the profile is saved in a memory, and further comprising retrieving the profile from the memory by a processor associated with the memory.

9. The method of claim 7, wherein the profile is a recommended profile based on at least one of a user height, a user weight, and user body measurements.

10. The method of claim 1, further comprising:
receiving input requesting an adjustment of the pressure within the at least one cushion;
obtaining second pressure measurements from the plurality of pressure sensors;
identifying a set of one or more inflatable chambers based at least in part on the second pressure measurements; and
adjusting the pressure of the set of one or more inflatable chambers.

11. The method of claim 10, wherein identifying the set of one or more inflatable chambers comprises identifying inflatable chambers associated with a highest measurement from the second pressure measurements.

12. The method of claim 1, wherein the at least one inflatable chamber is identified based at least in part on a medical condition associated with a user of the cushion.

13. The method of claim 1, wherein the at least one inflatable chamber is identified based at least in part on user preferences.

14. A human transport system comprising:
At least one cushion comprising a plurality of inflatable chambers;
a plurality of pressure sensors positioned on the at least one cushion;
one or more processors coupled to the plurality of pressure sensors and configured to receive pressure measurements from the plurality of pressure sensors and identify one or more inflatable chambers from the plurality of inflatable chambers based on the pressure measurements; and
an actuator coupled to the one or more processors and configured to adjust a pressure in the one or more inflatable chambers.

15. The human transport system of claim 14, wherein the human transport system comprises a wheelchair.

16. The human transport system of claim 14, wherein the at least one cushion comprises at least one of a seat cushion and a back cushion.

17. The human transport system of claim 14, wherein the one or more processors are configured to:
identify a profile saved in memory comprising pre-set pressure values for each of the plurality of inflatable chambers;
compare the pre-set pressure values for each of the plurality of inflatable chambers to the pressure measurements from the plurality of sensors; and
identify the one or more inflatable chambers where the pre-set pressure values do not match the pressure measurements.

18. A system for providing health care to a user comprising:
a human transport system comprising:
a cushion comprising a plurality of inflatable chambers;
a plurality of pressure sensors located on the cushion; and
one or more biometric sensors associated with the human transport system and configured to sense biometric data of a user of the human transport system;
a backend system comprising a memory; and
an electronic medical records system associated with a medical provider for a user of the human transport system,
wherein the human transport system is configured to adjust a pressure of one or more of the plurality of inflatable chambers based on pressure readings from the plurality of pressure sensors and to transmit the pressure readings and the biometric data of the user from the one or more biometric sensors to the backend system, and wherein the backend system is configured to at least one or store the pressure readings and the biometric data of the user and transmit the pressure readings and the biometric data of the user to the electronic medical records system.

19. The system of claim 18, wherein the one or more sensors comprise at least one of a blood oxygen sensor, a blood glucose sensor, a heart rate sensor, and a blood pressure sensor.

20. The system of claim 18, wherein the electronic medical records system is configured to receive the pressure readings and the biometric data of the user, receive information related to a treatment plan for the user, and transmit the information related to the treatment plan to the human transport system or backend system, wherein the treatment plan is developed by a medical provider based at least in part on the pressure readings or the biometric data of the user.
